(19) United States
(12) Patent Application Publication
(10) Pub. No.: US 2018/0304031 A1
(43) Pub. Date: Oct. 25, 2018

(54) MODIFIED NEBULIZER, METHOD AND SYSTEM FOR DELIVERING PHARMACEUTICAL PRODUCTS TO AN INDIVIDUAL

(71) Applicants: Nicholas A. Havercroft, Scunthorpe (GB); Donald M. Pell, Saint Petersburg, FL (US); Michael P. Spuza, Largo, FL (US); Govindan P. Nair, Seminole, FL (US)

(72) Inventors: Nicholas A. Havercroft, Scunthorpe (GB); Donald M. Pell, Saint Petersburg, FL (US); Michael P. Spuza, Largo, FL (US); Govindan P. Nair, Seminole, FL (US)

(21) Appl. No.: 16/025,437

(22) Filed: Jul. 2, 2018

Related U.S. Application Data
(63) Continuation-in-part of application No. 15/174,178, filed on Jun. 6, 2016.

(51) Int. Cl. A61M 11/00; A61K 9/00

(52) U.S. Cl. CPC A61M 11/003; A61K 31/465; A61M 11/005; A61K 9/0078

(57) ABSTRACT

A nebulizer comprising a nebulizing chamber with a vibrating mesh grid having a plurality of precision openings and said chamber with a liquid pharmaceutical product in a cartridge contacting the mesh grid for producing particle sizes of two (2) to four (4) microns; and an entrainment port for drawing air into the nebulizing chamber and through the grid. A control circuit, a source of electrical energy in said circuit such as two 1.5 volt lithium titanate batteries connected to said grid for producing vibration. A switch to turn said grid off and on and means for providing a first measured time period of four seconds for taking a first breath of air through the entrainment port and grid and through the thousands of passageways into the lungs and bloodstream of an individual. A second time period during which no vibration occurs. The grid is also self-cleaning as described.

### CROSS REFERENCE TO RELATED APPLICATIONS

The present application is a further improvement of an earlier filed U.S. patent application Ser. No. 15/174,178, filed on Jun. 6, 2016, and is hereby incorporated by reference in its entirety into the present application.

### FIELD OF THE INVENTION

This invention relates to an improved pulmonary delivery system, nebulizer, and pharmaceutical delivery system for delivering minute amounts of a pure pharmaceutical product disposed in very dilute solutions.

The use of the lung as a drug delivery avenue is relatively new. The lung has a surface area of a doubles tennis court and 100% of the cardiac output goes through it. Particles of an average size of about 2 microns pass through the alveoli and into the blood stream in about 7 seconds and 97% Of the particles are absorbed. The lung as a drug delivery system also avoids the “first pass effect” in which drugs given by the gastrointestinal tract pass through the liver and therefore the blood levels are reduced dramatically. Therefore, higher blood levels can be attained at a lower dosing concentration for the above reasons.

### BACKGROUND FOR THE INVENTION

Ultrasonic atomizers and nebulizers have been used for many years to atomize relatively small volumes of liquid such as approximately 50 ul to provide a high proportion of lung accessible droplets of less than 10 microns. For example, a U.S. Pat. No. 5,716,002 of Haack et al. issued Feb. 10, 1998 discloses an ultrasonic atomizer which includes an electrically excitable piezoceramic element and a coupling body being operatively connected to the piezoceramic element. The coupling body has a surface coming into contact with a liquid to be atomized and the surface is in the form of a cap-shaped protuberance. The cap-shaped protuberance and the coupling body are formed of a metallic solid material.

A more recent U.S. Patent of Ivri et al. U.S. Pat. No. 6,014,970 discloses a method and apparatus for storing chemical compounds in a portable inhaler. As disclosed, there is a cartridge that is coupled to a liquid dispenser and includes a substance in a dry state. A liquid is dispersed from the dispenser into the cartridge so that the substance may be dissolved into the dispersed liquid to form a solution. The solution is then transferred from the cartridge and onto an atomization member which is operated to aerosolize the solution.

Further, a method for transporting a liquid for atomization and a method and device for atomizing the same are disclosed in a U.S. Pat. No. 9,168,555 of Tsai et al. As disclosed, an ultrasonic nozzle device employing a design of cascaded multiple Fourier horns in resonance produce micrometer-sized monodispersed or narrowly-sized droplets with greatly reduced electrical drive power requirements. The liquid to be atomized is brought externally to or adjacent to the endface of a nozzle tip. The above liquid transport method is equally applicable to the ultrasonic nozzle-array devices that are made up of a plurality of ultrasonic single-nozzle devices configured in parallel. The longitudinal length, transverse width, shape, and area of the nozzle endface of a single-nozzle and nozzle-array devices may be tailored or designed to obtain optimum or large quantities of product droplets to achieve high throughput. By increasing the drive frequency to 8 MHz or higher, sub-micrometer-sized monodispersed or narrowly-sized droplets can be produced using the ultrasonic single-nozzle and nozzle-array devices or any solid endface.

In our earlier application, Ser. No. 15/174,178, filed Jun. 6, 2016, we disclosed a pulmonary delivery system for inhaling micron droplets of a liquid pharmaceutical product such as insulin, epinephrine or nicotine solution into an individual's lungs and into their bloodstream.

Notwithstanding the above, it is presently believed that there is a need and a potential commercial market for an improved Nebulizer, Method and System for Delivering a Pharmaceutical Product to a patient in accordance with the present invention.

### SUMMARY OF THE INVENTION

In essence, a first embodiment of the present invention contemplates a modified nebulizer and/or pulmonary delivery system for inhaling micron droplets of a liquid medical or pharmaceutical product such as an insulin, epinephrine or nicotine solution into an individual's lungs and into their bloodstream. In a preferred embodiment of the invention, the device or system comprises a nebulizer or modified nebulizer for producing a mass of micron droplets from a vibrating micro micron mesh grid made from a platinum or palladium plate referred to sometimes herein as “the grid” for producing a mist with particle sizes of about two (2) to four (4) microns. In a first form of the invention, the micron mesh grid includes about 4,800 precision holes or tapered passageways. The system and device also includes an elongated body having an openable and closeable housing for a cylinder for containing a replaceable cartridge or capsule.

The device employs a reverse piezoelectric principle in which a 3 volt current is passed through a palladium/platinum grid causing it to vibrate. When a solution is passed through this grid, it produces about two (2) micron particles which are readily absorbed into the blood stream.

Applicants further disclosed a system or device including an elongated body having an openable and closable portion therein and/or a cylinder for containing a replaceable cartridge as for example a capsule as opposed to a refillable container since refillable containers or cartridges have been avoided in view of the potential risk of spillage and lack of dosage control by a non-professional. In a preferred embodiment of the invention, a threaded bar coded or chip regulated capsule with a silicone cap is used. Once the cap is removed and the capsule is threaded securely to the grid a signal is sent signaling the device that the contents are acceptable for nebulization. The device or system also includes a mouthpiece for inhaling micron droplets of a nicotine solution into an individual's lungs, and into their bloodstream and a source of electric energy, and an electric input for vibrating the micron mesh grid.

A still further embodiment of the invention contemplates a portable medical inhaler for limiting the inspiration time of an initial inspiration and subsequently to prevent a second or subsequent inspiration for a preselected time following an initial or previous inspiration. The inhaler includes a tubular body having a cylinder for receiving a cartridge or capsule of a limited amount of three to five milliliters of a pharmaceutical product such as a relatively pure nicotine dissolved in a dilute saline solution. To be more specific, relatively pure nicotine is addictive and in excess it is a toxic material. It is not generally believed to be a dangerous substance in small amounts. However, the tars and chemicals associated with smoking cigarettes are believed to be a carcinogen to a variety of organs and smokers particularly heavy smokers experience withdrawal symptoms within two hours after a last smoking experience.

However, it has now been recognized that inhaling very small amounts of small particles of a nicotine saline solution can be helpful to avoid withdrawal symptoms. Nevertheless, it is important to prevent the inhaler from delivering an excess amount of nicotine particularly for children and the potential death of pets due to the ingestion of one or more cartridges or capsules. Accordingly, an inhaler for permitting an amount of an initial inspiration and to prevent a second or subsequent inspirations for a preselected time following an initial or previous inspiration is provided. It is also important to limit the inhaler to use replaceable cartridges as opposed to refillable containers to avoid having relatively untrained individuals working with toxic materials and exposing the nicotine solution to contamination, bacteria, fungus and yeast.

The commonly used open nebulizers do not accurately dose medications because a patient inhales for 1 to 3 seconds and exhales for 3 to 4 times longer, therefore wasting a significant portion of the nebulized solution as this type of device produces a continuous flow. By using an on off switch and an accurately dosed cartridge, flow of medication only occurs in one direction into the patient. Applicants' device also avoids reservoir type designs to prevent mixing errors, and to avoid the risk of contamination of the liquids and the device by bacteria, yeast or fungi. These are common problems with other open designs.

A further embodiment of the invention includes a medical product, and a nebulizer for reducing and/or eliminating withdrawal symptoms from using tobacco products. The pharmaceutical product comprises a mixture of between about 97% and about 99¾% of a liquid saline solution and between one to three milligrams of nicotine. In addition, the above two compounds can further include an addition of up to about 3% of a flavor. A preferred form of this embodiment includes about 98½% saline solution and about one to three milligrams of relatively pure nicotine. Like the aforementioned product a preferred form consist essentially of 98½% saline solution and about one to three milligrams of nicotine without the tars and chemicals associated with burning or heating tobacco products. Using this device, no heating of liquid occurs.

These amounts of nicotine, 1 to 3 milligrams, are those amounts usually attained by smokers using cigarettes which contain 6 to 18 milligrams of nicotine. The superior drug delivery of Applicants' device allows for a much smaller dose of nicotine which is another safety feature.

In a fourth embodiment of the invention, a drug delivery system or device includes a microprocessor, a microchip in a circuit for limiting an initial inspiration to two to four seconds of vibration for producing micron droplets and a subsequent or second period of time such as a period of 15 seconds without any vibration. This is generally accomplished by timing a discontinuous or prevention of vibration of the micron mesh grid by interrupting a connection between a source of electric power and the vibratable micro micron mesh grid. Even though an individual may continue to inhale he/she will receive only air from the entrainment port and/or any posterior entrainment port for an inhaled breath in excess of four seconds.

In essence, the present invention discloses a nebulizer comprising a nebulizing chamber including a vibratable micron mesh grid having a plurality of precision openings therethrough disposed within the chamber. The nebulizer also includes a liquid pharmaceutical product contained in the nebulizing chamber and passing through the openings in the vibratable micro micron mesh grid for producing particle sizes of the liquid pharmaceutical product of about two (2) to about three (3) microns; and an entrainment port for drawing air and liquid pharmaceutical product into and through the grid. The grid is vibrated at about 120,000 vibrations per second and a switch is provided to automatically turn the vibration off following a four (4) second period of inspiration that is followed by a 15 second period of no vibration, to allow an individual time to exhale. This second period also prevents an excess of nicotine from being inhaled. Other provisions are provided for the safety of an individual, children and inadvertent ingestion of one or more capsules by a family pet.

To be more specific, a preferred embodiment of the present invention contains a one to three milligram solution of nicotine in a saline solution. A further embodiment of the invention provides capsules containing one (1) to three (3) milligrams of nicotine. Still further, the nebulizer according to a further embodiment of the invention contains a new capsule in a second closed compartment to be moved to the nebulizing chamber when it is time for the next treatment, much like scuba tanks.

A circuit for providing an inspiration period of two-four seconds followed by a period to exhale of about 15 seconds. Other embodiments are as follows, display indicating battery life, time to clean the grid and other notifications. The time and date may be presented on an LED screen. In a preferred embodiment of the invention, a self-cleaning grid is programmed in the device at the start of each treatment.

The grid may be cleaned by depressing the start button for five seconds causing the grid to ultra-vibrate prior to each use. This mechanically cleans the grid effectively.

In essence, a nebulizer in accordance with a preferred embodiment of the invention comprises and/or consists of a nebulizing chamber including a vibratable micro micron mesh grid having a plurality of precision openings therethrough as for example 4,000 tapered passageways that end in a few thousand or more openings for forming particle sizes of about two microns and a comparable number of tapered passageways that diminish in diameter as it reaches the surface of a grid for producing particle sizes of about 2 microns plus a liquid pharmaceutical product contained in the nebulizing chamber and providing an inhalation through the openings in the vibratable micro micron mesh grid for producing particle sizes of the liquid pharmaceutical product of about two (2) to four (4) microns. In addition, the preferred embodiment includes an entrainment port for drawing air and liquid pharmaceutical product into and through the grid and into the lungs and bloodstream of an individual.

A variety of grids may be used, depending on the characteristics of the solution to be nebulized. These grids are produced by the Tanaka Corporation of Japan. The grid is fixed in each device and matches the medicine to be nebulized.

A nebulizer comprising a nebulizing chamber including a vibratable micro micron mesh grid having a plurality of precision openings therethrough disposed in said nebulizing chamber; further the preferred embodiment of the invention includes a liquid pharmaceutical product contained in the nebulizing cartridge and passing through the openings in the vibratable micro micron mesh grid for producing particle sizes of the liquid pharmaceutical product of about two (2) to four (4) microns and an entrainment port having an opening with a diameter of about ⅛ inch.

In the preferred embodiment, the nebulizer includes a control circuit, a source of electrical energy as for example two rechargeable 1½ volt lithium titanate batteries for vibrating the grid at about 120,000 vibrations per second and a switch to turn the grid on and off. A further feature of a preferred embodiment is a programmable switch for turning the grid on and off with an initial time period of about four seconds and immediately followed by a 15 second delay for allowing the patient time to exhale.

The invention will now be described in connection with the accompanying drawings wherein like numbers are used to indicate like elements.

### DESCRIPTION OF THE DRAWINGS

FIG. 1 is a front end view of a nebulizer in accordance with a first embodiment of the invention;

FIG. 2 is an end view of a first portion of the back surface of the nebulizer illustrated in FIG. 1;

FIG. 3 is a schematic side view of the nebulizer shown in FIGS. 1 and 2; and

FIG. 4 is a schematic top projection of a top view of the nebulizer shown in FIGS. 1-3.

### DESCRIPTION OF THE PREFERRED EMBODIMENTS OF THE INVENTION

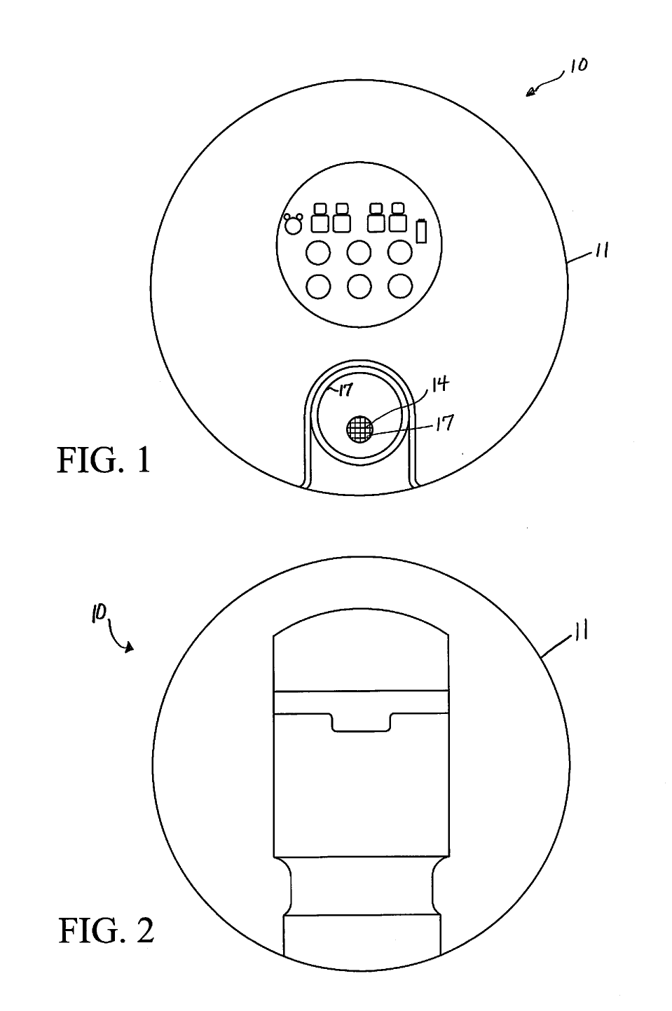

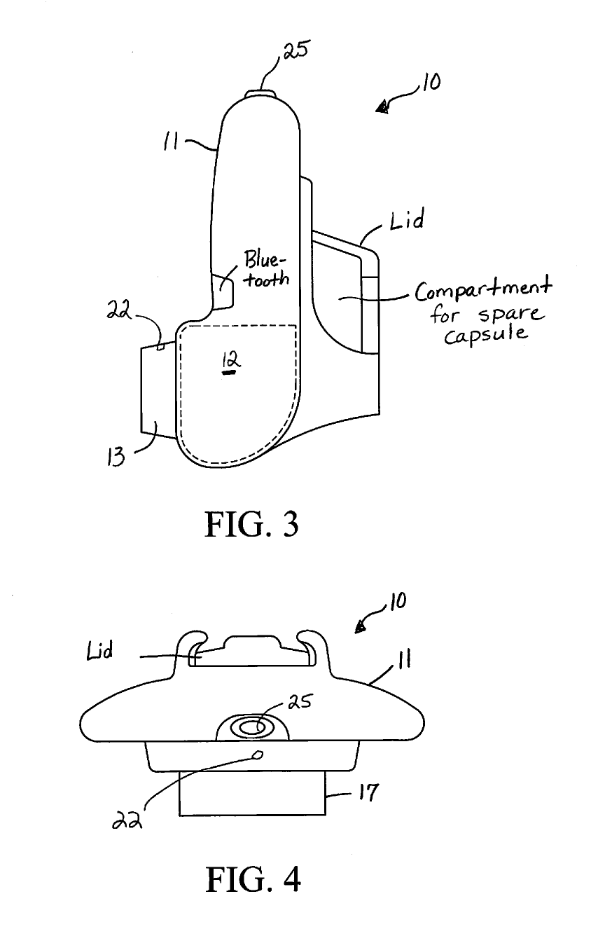

In essence, a nebulizer 10 in accordance with a first embodiment of the invention comprises or consists of an outer housing or body 11 including a nebulizing chamber 12 shown by dotted lines in FIG. 3 and includes a vibratable micro micron mesh grid 16 visible through the mouthpiece 17 in FIG. 1. The plurality of precision openings and/or tapered passageways 16 therethrough are disposed in the nebulizing chamber 12 and indicated by the openings 16 within the grid 14. A liquid pharmaceutical product 18 is also contained in the nebulizing chamber 12 and is drawn together with air through the grid 14 which is vibrated for producing particle sizes of the liquid pharmaceutical product of about two (2) to about four (4) microns.

The entrainment port 22 in said body 11 and an in an upper portion of the mouthpiece 17 allows an individual to draw air into the nebulizing chamber 12 through the grid 14 and into the lungs of a patient. The nebulizer 10 also includes a control circuit, and a source of electrical energy for vibrating the grid together with a programmable switch to turn the grid on and off and means for including a first measured time period of about four seconds of time for drawing a deep breath of air through the mouthpiece 17 and through the entrainment port 22 through the grid 17′ and into an individual's lungs. The means for establishing a first time period of about four seconds during which an individual draws a deep breath through the entrainment port through the micro micron mesh grid and into a patient's lungs. This first period establishes the means for establishing a second period of time for about fifteen (15) seconds in which the micro micron mesh grid is not vibrated to allow time for a patient to exhale. The patient's inhaled breath is the sole means for creating an airflow through the device. Further, Applicants' device does not have any mechanism for heating the airflow or pharmaceutical product.

A cartridge compartment, lid and power switch are also shown. Further a connection for Bluetooth or Wi-Fi is shown above the mouthpiece in a forward portion of the body 11. Further at the top of the nebulizer 10 is a power switch 25 for activating the nebulizer 10.

In essence, the first embodiment of the invention comprises or consists of a nebulizer body and a nebulizing chamber disposed in the nebulizer body and an entrainment port, a mouthpiece and a liquid pharmaceutical product for drawing air through said entrainment port through said mouthpiece and into said nebulizing chamber and wherein said nebulizing chamber includes a vibratable micro micron mesh grid made from a platinum or palladium plate and containing thousands of precision tapered passageways through the plate and ending in thousands of openings in an opposite side of the plate. The producing particle sizes of the pharmaceutical product are made in sizes of about two (2) to four (4) microns of the pharmaceutical product and an electrical circuit and a source of electrical energy connects the grid and the source of electrical energy for a period of about two (2) to four (4) seconds and immediately thereafter disconnecting the grid from the source of electrical energy for a period of 15 seconds to allow a user to exhale during a period of no vibration to the grid.

A second embodiment of the invention incorporates a liquid pharmaceutical product of relatively pure nicotine in a dilute solution containing about 1% pure nicotine and water or saline up to about 3 milligrams of pure nicotine and 99¾% of water or saline plus up to about 3% flavor.

A third embodiment of the invention includes a liquid pharmaceutical capsule containing between 1 and 3 milligrams of nicotine therein.

In a further embodiment of the invention the liquid pharmaceutical capsule contains between about 1 and 3 milligrams of nicotine.

In a still further embodiment of the invention the nebulizer includes a second closed compartment for containing a second capsule of a liquid pharmaceutical product.

In a further embodiment of the invention the nebulizer contains means for opening a capsule providing an opening at each end at or near each end of said capsule to allow an airstream to be drawn through said capsule.

The nebulizer according to a further embodiment of the invention includes an LED display and a program to telephone a user's smartphone and indicate that it is time to take medication.

In addition, an embodiment of the invention includes a face display including a flowrate indicator and a warning to clean said grid and a further warning to change batteries and to replace the capsule.

A still further embodiment of the invention includes a micro USB port for charging the lithium titanate batteries.

An additional nebulizer according to claim 1 which includes an extra disposable capsule to allow disposal capsules for accurate dosing and reducing the risks of contamination.

Still further, the nebulizer according to claim 1, which includes means for limiting an initial period of time during which a switch in said electric circuit connects said nebulizing grid to a source of three volts provided by two 1.5 volt lithium titanate batteries for a first period of time for about four seconds and a second period of time of about 15 seconds immediately following the first period of time.

Another embodiment of the invention provides a nebulizer in which said first period of time of about three (3) to four (4) seconds and a second period of time for at least 15 seconds.

An additional embodiment of the invention contemplates a nebulizer in which the nebulizer includes a three speed period of electrification of the nebulizing grid selectable by a user.

A still further embodiment of the invention contemplates a nebulizer in which said three-speed indicator includes a slow speed, a medium speed and a fast speed.

A further embodiment of the invention includes a nebulizer that delivers 2-4 micron particles into the lungs and the bloodstream and avoids the heat effect to attain higher bloodstream levels at lower concentrations of medical products.

A next to final nebulizer according to claim 1, in which the nebulizer delivers 2-4 micron particle sizes into the lungs and bloodstream and avoids the “first pass effect” to attain higher blood levels at lower concentration of products.

Finally, a nebulizer in which the grid is cleaned by depressing the on/off switch for a period of five seconds which automatically cleans the grid by ultra-vibration prior to each nebulizer use.

While the invention has been described in connection with its preferred embodiments it should be recognized that changes and modifications may be made therein without departing from the scope of the appended claims.

What is claimed is:

1. A nebulizer comprising:
a nebulizer body and a nebulizing chamber disposed in said nebulizer body;
an entrainment port, a mouthpiece and a liquid pharmaceutical product for drawing air through said entrainment port through said mouthpiece and into said nebulizing chamber; and
wherein said nebulizing chamber includes a vibratable micro micron mesh grid made from a platinum or a palladium plate and containing thousands of precision tapered passageways through said plate and ending in thousands of openings in an opposite side of said plate for producing particle sizes of said pharmaceutical product in sizes of about two (2) to four (4) microns of said pharmaceutical product; and
an electric circuit and a source of electrical energy connecting said grid to said source of electrical energy for a period of about three-four seconds and immediately thereafter disconnecting said grid from said source of electrical energy for a period of 15 seconds to allow a user to exhale during a period of no vibration to said grid.

2. The nebulizer according to claim 1, in which said liquid pharmaceutical product is pure nicotine in a dilute solution containing about one percent pure nicotine and 99% water or saline to 3 milligrams pure nicotine and 99¾% of water or saline and up to 3% flavor.

3. The nebulizer according to claim 1, in which said liquid pharmaceutical capsule contains between one and three milligrams of nicotine.

4. The nebulizer according to claim 2, which contains a second closed compartment for containing a second capsule of a liquid pharmaceutical product.

5. The nebulizer according to claim 2, in which said means for opening a capsule provide an opening at each end of said capsule to allow an airstream to be drawn through said capsule.

6. The nebulizer according to claim 5, which includes an LED display and a program to telephone a user's smartphone and indicate that it is time to take medication.

7. The nebulizer according to claim 5, which includes a face display including a flow rate indicator, a warning to clean said grid, a warning to change batteries and a warning to replace a capsule.

8. The nebulizer according to claim 5, which includes a micro USB port for charging the lithium titanate batteries.

9. The nebulizer according to claim 5, which includes an extra disposable capsule to allow disposable capsules for accurate dosing and reduces the risk of contamination.

10. The nebulizer according to claim 5, in which all plastic parts are free of bisphenol A.

11. The nebulizer according to claim 5, which includes means for limiting an initial period of time during which a switch in said electric circuit connects said nebulizing grid to a source of electricity for a first period of time and disconnecting said nebulizing grid from said source of electricity for a second period of about 15 seconds immediately following said first period of time.

12. The nebulizer according to claim 11, in which said first period of time is three (3) to four (4) seconds and said second period of time is at least fifteen (15) seconds.

13. The nebulizer according to claim 5, in which said nebulizer includes a three speed period of electrification of said nebulizing grid selectable by said user.

14. The nebulizer according to claim 13, in which said three-speed indicator includes a slow speed, a medium speed and a fast speed.

15. A nebulizer which delivers 2-4 micron particles into the lungs and the bloodstream and avoids the heat effect to attain higher bloodstream levels at lower concentrations of products.

16. The nebulizer according to claim 15, in which a nebulizer delivers 2-4 micron particles into the lungs and bloodstream and avoids the “first pass effect” to attain higher blood levels at lower concentration of products.

17. A mesh nebulizer in which the grid is cleaned by depressing the on/off button for a period of five seconds which cleans the grid by ultra-vibration prior to each nebulizer use.
